(19) United States
(12) Patent Application Publication
(10) Pub. No.: US 2025/0172576 A1
(43) Pub. Date: May 29, 2025

(54) METHOD FOR DETERMINING CHEMICAL STRUCTURE OF LIPID AND ION MOBILITY-TANDEM MASS SPECTROMETER

(71) Applicant: SHIMADZU CORPORATION, Kyoto-shi (JP)

(72) Inventors: Wenjian SUN, Shanghai (CN); Yuling Li, Shanghai (CN); Xiaoqiang ZHANG, Shanghai (CN); Kang GUO, Shanghai (CN)

(21) Appl. No.: 18/958,015

(22) Filed: Nov. 25, 2024

(30) Foreign Application Priority Data
Nov. 27, 2023 (CN) 202311603399.8

(51) Int. Cl. G01N 33/92; G01N 27/623; G01N 33/68

(52) U.S. Cl. CPC G01N 33/92; G01N 27/623; G01N 33/6851; G01N 2458/20

(57) ABSTRACT

The present disclosure relates to the field of mass spectrometry, and particularly provides a method for determining a chemical structure of a lipid and an ion mobility-tandem mass spectrometer. The method for determining a chemical structure of a lipid includes: an ionization step of ionizing a sample to obtain sample ions; an ion mobility-based separation step of separating target lipid ions from the sample ions based on ion mobility; a first dissociation step of dissociating the target lipid ions with dissociation energy adjusted to break a first chemical bond of the target lipid ions; a mass-based selection step of selecting the target lipid ions, whose first chemical bond is broken, based on a mass number to obtain fragment ions; a second dissociation step of dissociating the fragment ions to at least break a second chemical bond of the fragment ions which has bond energy higher than the first chemical bond, to obtain diagnostic ions; and a mass analysis step of performing a mass analysis on the diagnostic ions.

### TECHNICAL FIELD

The present disclosure relates to the field of mass spectrometry, in particular to a method for determining a chemical structure of a lipid and an ion mobility-tandem mass spectrometer.

### BACKGROUND ART

Lipids are important nutrients and components of biological cells, and are closely related to some important immune functions and metabolic defects. The lipid metabolites and pathways strategy (LIPID MAPS) has been launched to establish a classification database to promote lipidomics research.

Complete labeling and identification information on lipids includes class, elemental composition, size and position of R-groups, the number and position of double bonds, and cis-trans isomeric orientation of double bonds. In addition, for unsaturated lipids with a glycerol backbone, such as glycerolipids and glycerophospholipids, the sn-position in the fatty acyl chain containing a carbon-carbon double bond also needs to be determined.

Tandem mass spectrometry plays an important role in the structural analysis of compounds. Hsu and Turk have proposed a pseudo MS3 tandem mass spectrometry method to identify the sn-position of glycerophospholipids. First, the in-source CID is used to obtain signals of the high-abundance fragment ions [M+Li−183]⁺ without head group, and then the fragment ions were subjected to collision-induced dissociation to produce the sn-diagnostic ions (J. Am. Soc. Mass Spectrom. 2003, 14, 352). For unsaturated lipids with a glycerol backbone, in order to simultaneously identify the double bond position and the sn-position in a single injection, Brodbelt et al. have provided a hybrid MS3 method of combining collision activation with ultraviolet photodissociation in the article “Pinpointing Double Bond and sn-Positions in Glycerophospholipids via Hybrid 193 nm Ultraviolet Photodissociation (UVPD) Mass Spectrometry” (J. Am. Chem. Soc. 2017, 139, 15681-15690), but the abundance of diagnostic ions still needs to be further improved. These pseudo MS3 and MS3 analysis methods are not good at isomer selection and analysis. In addition, when target ions are selected by quadrupole, non target ions will be lost, so that the overall duty cycle is low. In addition to directly performing multi-stage tandem mass spectrometry analysis on compounds, chemical derivatization can also be used to pre-modify analytes to increase ionization efficiency, increase structural differences, improve chromatographic behavior, etc., and then multi-stage tandem mass spectrometry analysis is performed on derivatives. In the article “Large-scale lipid analysis with C═C location and sn-position isomer resolving power” (Nat. Commun., 2020, 11, 375), Ma et al. use charge-tagging derivatization and MS3 to accurately identify the C═C position and the sn-position of derivatized glycerophospholipids. The method provided in this article requires the use of an ion trap mass spectrometer to select specific precursor ions based on m/z, and the duty cycle and resolution are limited by the ion trap mass spectrometer.

High-throughput separation and analysis of isomers can be achieved using ion mobility spectrometry under gas phase conditions. When combined with mass spectrometry, the system has a high resolution and has a similar effect to multi-stage tandem mass spectrometry (Biochem. Soc. T. 2020, 48, 2457; Anal. Chem. 2006, 78, 4161). The ion mobility spectrometry is used to obtain detailed structural information on analytes and has been widely used in structural identification in metabolomics, glycomics, and proteomics. For example, a large amount of research has reported that the separation and de novo measurement of carbohydrate isomers can be achieved by a combination of cyclic ion mobility (cIM) and collision-induced dissociation (CID), such as a cIM-CID-cIM mode and a cIM-CID-cIM-CID-cIM mode (Anal. Chem. 2021, 93, 6254; Annual Rev. Anal. Chem. 2023, 16, 27). The Bleiholder team has proposed a scheme of combining tandem ion mobility spectrometry and tandem mass spectrometry based on trapped ion mobility spectrometry (TIMS), which has functions such as mobility-based selection and collision activation (U.S. Pat. No. 10,794,861B2, Analyst 2022, 147, 2317), and by which the structural identification of peptides and proteins (Analyst 2018, 143, 2249; J. Am. Soc. Mass Spectrom. 2023, 34, 2247), sugars and their isomers thereof are achieved (Anal. Chem. 2023, 95, 747). Nicholas B. Borotto and colleagues have achieved collision-induced unfolding prior to mobility selection based on TIMS, which can quickly distinguish the conformation of proteins (J. Am. Soc. Mass Spectrom. 2022, 33, 83). In addition, the pre-mobility collision-induced unfolding strategy can be further combined with tandem mass spectrometry to achieve precise protein sequencing (J. Am. Soc. Mass Spectrom. 2023, 34, 2232).

In recent years, good results in the in-depth structural identification of lipids have also been obtained by ion mobility mass spectrometry (J. Chromatogr. A, 2017, 1530, 90; J. Sep. Sci. 2018, 41, 20; Front. Mol. Biosci. 2023, 16, 10, 1112521). Baker and his colleagues have proposed a lipidomics analysis method in which reversed-phase liquid chromatography is combined with ion mobility mass spectrometry, which can separate lipid isomers in three dimensions: polarity, structure and size, mass-to-charge ratio, and also increase the peak capacity. The separation of different types of lipids can be achieved by liquid chromatography, and different classes of lipids form different ion trend lines in the ion mobility spectrometry. It should be noted that the poor separation of different lipid subclasses can also be observed in ion mobility spectrometry (Analyst, 2016, 141, 1649). Due to the limitation of instrument resolution, different lipid isomers can only achieve shoulder separation. F. Fernandez-Lima et al. used high-resolution TIMS under specific instrument parameters (average resolution exceeding 320) to identify C═C positional isomers of sodiate phosphatidylcholine and geometric isomers of protonate adducts. In addition, when the instrument parameters satisfy the ultra-high resolution (exceeding 410) conditions, this method can be used for sn-position isomerization identification (Anal. Chem. 2019, 91, 5021). However, the resolution of most commercial ion mobility instruments is less than 200, which makes it difficult to satisfy the above conditions. The formation of metal ion adducts is beneficial for increasing isomer resolution. M. Groessl et al. have used drift-time ion mobility mass spectrometry to distinguish double-bond positional isomerism, double-bond cis-trans isomerism and sn-isomerism of silver ion adducts of phosphatidylcholine (Analyst, 2015, 140, 6904). Similarly, Yan et al. have used drift-time ion mobility mass spectrometry to separate and analyze the C═C geometric isomers of monovalent copper ion adduct of phosphatidylcholine, but the resolving power is low (Int. J. Mass Spectrom. 2022, 479, 116889).

Although many methods for identifying lipid isomers based on ion mobility spectrometry have been developed, these methods require lipid standards as references, making it difficult to identify the structures of unknown compounds. In addition, the resolution of ion mobility spectrometry is not as good as that of mass spectrometry, which is limited in terms of the analysis of complex matrix samples. The combination of the separation ability of ion mobility and the resolving power of tandem mass spectrometry has great potential for the structural analysis of unknown lipid compounds in biological samples. The Brodbelt team has combined UVPD with the drift-time ion mobility mass spectrometry to successfully measure the CCS of lipid isomers, and identify the positions of C═C and cyclopropanes of lipids (Anal. Chem. 2022, 94, 4252). However, this method is only suitable for the analysis of polar lipids, and is less likely to detect low-polar lipids. Xia et al. have combined the P—B reaction and trapped ion mobility tandem mass spectrometry to successfully separate and analyze conjugated fatty acid isomers. The mobility characteristic peaks can be accurately attributed to double bonds in the absence of reference materials by combining an ion mobility spectrum with a tandem mass spectrum. However, the reaction products of the PB reaction with conjugated fatty acids are diverse and the resulting mobility spectra are highly complex, which limits application in complex mixtures (Anal. Chem. 2019, 91, 7173). In 2023, Xia's team has combined liquid chromatography, ion mobility spectrometry, and the P—B reaction tandem mass spectrometry to establish a deep lipid structure analysis process, which can identify the double bond position and sn-position in steps. The process has been successfully used for lipidome analysis in biological samples such as bovine liver and cells to obtain more complete lipid profile information (Nat. Commun. 2023, 14, 4263). However, the step-by-step pre-treatment and multi-batch analysis process not only reduce the analysis throughput, but also cause sample loss.

### SUMMARY OF THE INVENTION

In view of the above problems, the present disclosure provides a method for determining a chemical structure of a lipid and an ion mobility-tandem mass spectrometer, which can improve the abundance of diagnostic ions and have a good duty cycle and resolution.

According to a first aspect of the present disclosure, there is provided a method for determining a chemical structure of a lipid. The method includes:
an ionization step of ionizing a sample to obtain sample ions;
an ion mobility-based separation step of separating target lipid ions from the sample ions based on ion mobility;
a first dissociation step of dissociating the target lipid ions with dissociation energy adjusted to break a first chemical bond of the target lipid ions;
a mass-based selection step of selecting the target lipid ions, whose first chemical bond is broken, based on a mass number to obtain fragment ions;
a second dissociation step of dissociating the fragment ions to at least break a second chemical bond of the fragment ions which has bond energy higher than the first chemical bond, to obtain diagnostic ions;
a mass analysis step of performing a mass analysis on the diagnostic ions.

Optionally, the lipid is an unsaturated lipid having several carbon-carbon double bonds in a fatty acyl chain, and the method is used for identifying a position of the carbon-carbon double bond in the fatty acyl chain and the sn-position of the fatty acyl chain.

Optionally, the method further includes, before the ionization step, a derivatization reaction step of labeling the carbon-carbon double bond using a derivatization reaction.

Optionally, the lipid is a phospholipid or a sphingolipid, the first chemical bond is a polar head group of the phospholipid or a polar head group of the sphingolipid, and the second chemical bond is a chemical bond generated from C═C derivatization.

Optionally, the derivatization reaction is an aziridination reaction, an epoxidation reaction, a Paternò-Buchi reaction, a singlet oxygen-ene reaction, or a Diels-Alder reaction.

Optionally, the lipid is a fatty acid, a glycerolipid, a glycerophospholipid, a sphingolipid, a sterol lipid, a prenol lipid, a saccharolipid, or a polyketide.

Optionally, the method further includes: a first pre-scan step of performing a mass analysis on the sample ions that are not subjected to the first dissociation step and the second dissociation step.

Optionally, the method further includes: a second pre-scan step of performing a mass analysis on the sample ions that are subjected to only one dissociation.

According to a second aspect of the present disclosure, there is provided an ion mobility-tandem mass spectrometer including an ion source, an ion mobility spectrometer, a first dissociation device, a mass filter, a second dissociation device, and a mass analyzer. The ion source ionizes a sample to obtain sample ions. The ion mobility spectrometer separates target lipid ions from the sample ions. The first dissociation device dissociates the target lipid ions, in which dissociation energy is adjusted to break a first chemical bond of the target lipid ions, thereby obtaining desired fragment ions. The mass filter selects the target fragment ions. The second dissociation device dissociates the fragment ions to at least break a second chemical bond of the fragment ions which has bond energy higher than that of the first chemical bond to obtain diagnostic ions. The mass analyzer performs a mass analysis on the diagnostic ions.

Optionally, the first dissociation device is a collision-induced dissociation device and has a terminal electrode voltage of 10 eV to 70 eV, and the second dissociation device is a collision-induced dissociation device and has dissociation energy of 30 eV to 70 eV.

### Beneficial Effects

Precise ion selection: Various target lipid ions are separated using the high duty cycle of ion mobility and then are used for subsequent multiple targeted tandem mass spectrometry analysis to reduce the complexity of a single spectrum. Pseudo-multistage collision-induced dissociation: The first chemical bond with weaker bond energy of the target lipid ions is first broken to remove an interfering groups bonded by the first chemical bond in the target lipid ions, and then the fragment ions from which the interfering groups are excluded are selected and subjected to secondary dissociation, so that the mass spectrometry signal intensity of the diagnostic ions finally generated can be improved, the problem of increased spectrum complexity caused by the peaks related to interfering groups appearing in the final mass spectrum can be alleviated or avoided, and the spectrum is more simplified to facilitate the analysis and determination of the chemical structure of lipids.

### DESCRIPTIONS OF THE DRAWINGS

FIG. 1 is a flow chart of a method for determining a chemical structure of a lipid provided in an embodiment of the present disclosure.

FIG. 2 is a schematic diagram of a system for implementing the method for determining a chemical structure of a lipid provided in the embodiment of the present disclosure.

FIG. 3 is a schematic diagram of a structure of a more preferred ion mobility-tandem mass spectrometer provided in the embodiment of the present disclosure.

FIG. 4 shows a specific reaction process of PC (18:1/16:0) in the present embodiment.

FIG. 5 is a schematic diagram illustrating why a sn-position of a carbon-carbon double bond can be identified based on diagnostic ions with a mass number of m/z=290 or 360 [M+Na⁺].

FIG. 6 is a comparison chart of measurement results obtained by the common tandem mass spectrometry MS2 method in the prior art and measurement results obtained by the method provided in the embodiment of the present disclosure.

### LIST OF REFERENCE NUMERALS

1: ion source; 2: ion mobility spectrometer; 3: first dissociation device; 4: mass filter; 5: second dissociation device; 6: mass analyzer; and 7: ion optical device.

### DETAILED DESCRIPTION OF THE EMBODIMENTS

The technical scheme in the embodiments of the present disclosure will be clearly and completely described below with reference to the accompanying drawings in the embodiments of the present disclosure. Obviously, the described embodiments are merely some, not all, of the embodiments in the present disclosure. Based on the embodiments in the present disclosure, all other embodiments obtained by those skilled in the art fall within the scope of the present disclosure.

A method for determining a chemical structure of a lipid provided in the present embodiment can be applied to identify lipids with interfering groups (such as polar head groups). The lipid may be, for example, a fatty acyl, a glycerolipid, a glycerophospholipid, a sphingolipid, a sterol lipid, a prenol lipid, a saccharolipid, or a polyketide.

In the method for determining a chemical structure of a lipid provided in the present embodiment, the first chemical bond with weaker bond energy of the target lipid ions is first broken to remove an interfering group connected by the first chemical bond in the target lipid ions, and then the fragment ions from which the interfering groups are excluded are selected and subjected to secondary dissociation. Through the above manner, the mass spectrometry signal intensity of the diagnostic ions finally generated can be improved, the problem of increased spectrum complexity caused by the peaks related to interfering groups appearing in the final mass spectrum can be alleviated or avoided, and the spectrum is more simplified to facilitate the analysis and determination of the chemical structure of lipids.

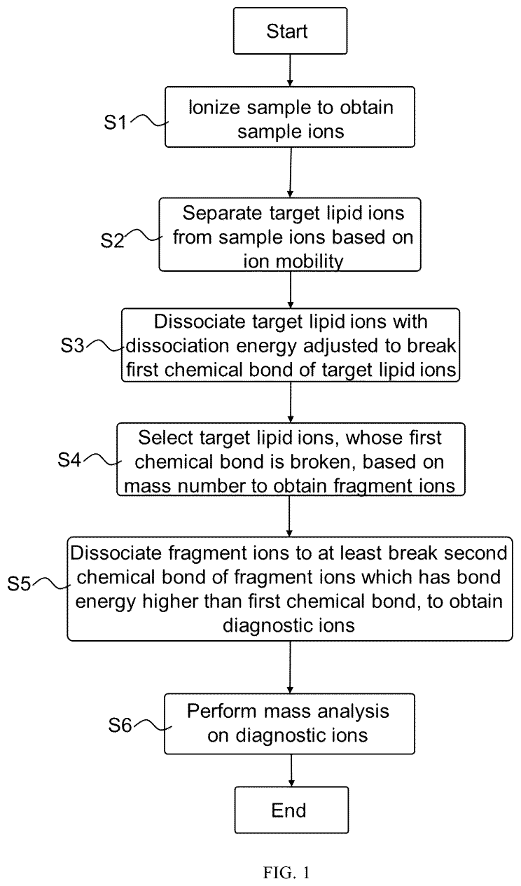

FIG. 1 is a flow chart of a method for determining a chemical structure of a lipid provided in the first embodiment of the present disclosure. Referring to FIG. 1, the method provided in the present embodiment includes an ionization step S1, an ion mobility-based separation step S2, a first dissociation step S3, a mass-based selection step S4, a second dissociation step S5, and a mass analysis step S6, in a sequential manner.

### Ionization Step S1

A sample is ionized to obtain sample ions.

### Ion Mobility-Based Separation Step S2

Target lipid ions are separated from the sample ions based on ion mobility.

### First Dissociation Step S3

The target lipid ions are dissociated with dissociation energy adjusted to break a first chemical bond of the target lipid ions.

The first dissociation of the target lipid ions, i.e., the first dissociation step S3, can selectively break the chemical bonds of the target lipid ions that have lower bond energy and may eventually generate interference signals (i.e., the first chemical bond connected to the interfering groups).

The first dissociation step S3 is a selective dissociation step, that is, when the first chemical bond is broken, the integrity of other chemical bonds (chemical bonds of a main chain, especially the second chemical bonds) is maintained as much as possible. Specifically, the above requirement can be satisfied by setting the dissociation energy to be slightly higher than a threshold value at which the first chemical bond can be broken. In some embodiments, when the first chemical bond has the lowest bond energy in the target lipid ions, the dissociation energy can be set to mainly break the first chemical bond and maintain the integrity of other chemical bonds to the greatest extent.

By setting the dissociation energy to be equal to or slightly higher than the threshold value at which the first chemical bond is broken, the first chemical bond can be selectively broken, so that the interference of interfering groups on the final mass spectrometry test results can be eliminated with less loss of ion abundance.

### Mass-Based Selection Step S4

The target lipid ions, whose first chemical bond is broken, are selected based on a mass number to obtain fragment ions.

Selective breaking of the first chemical bond can generate at least a pair of ions, one is an ion with an interfering group (such as a polar head group) and the other is an ion without an interfering group. The fragment ions selected in the mass-based selection step S4 are fragment ions from which the interfering groups connected by the first chemical bond are excluded, that is, ions without interfering groups.

### Second Dissociation Step S5

The fragment ions are dissociated to at least break a second chemical bond of the fragment ions which has bond energy higher than the first chemical bond, to obtain diagnostic ions.

The obtained fragment ions without interfering groups are further dissociated by the second dissociation step, that is, the second dissociation step S5. The second dissociation step S5 may be selective or non-selective, but no matter whether the second dissociation step S5 is selective or non-selective, too many spectral peaks associated with the interfering groups will not appear in a mass spectrum because the interfering groups have been removed in the first dissociation step S3 and the mass-based selection step S4, resulting in fewer spectral peaks in the mass spectrum and a stronger peak intensity of the mass peak of the diagnostic ion.

Generally speaking, in the case where the same type of dissociation device is used, the dissociation energy used in the second dissociation step S5 is higher than that used in the first dissociation step S3, so that chemical bonds with higher bond energy can be broken.

### Mass Analysis Step S6

A mass analysis is performed on the diagnostic ions.

### <Ion Mobility-Tandem Mass Spectrometer>

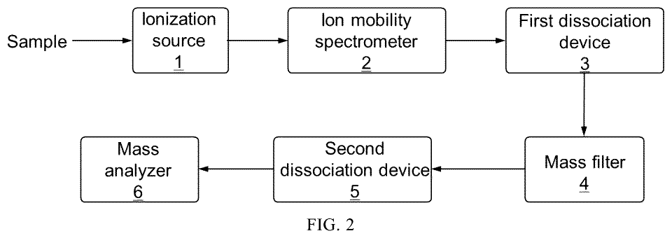

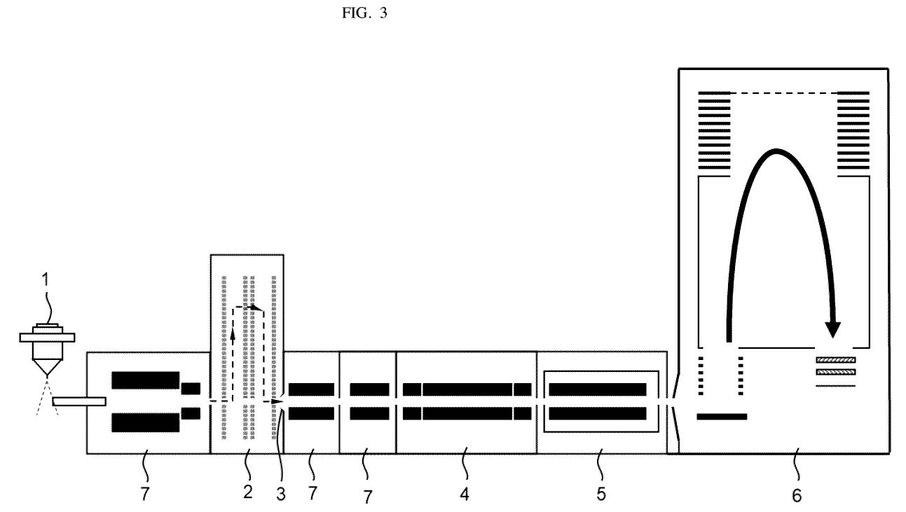

FIG. 2 is a schematic diagram of a system for implementing a method for determining a chemical structure of a lipid provided in the present embodiment. FIG. 3 is a schematic diagram of a structure of a more preferred ion mobility-tandem mass spectrometer provided in the present embodiment.

The ion mobility-tandem mass spectrometer includes an ion source 1, an ion mobility spectrometer 2, a first dissociation device 3, a mass filter 4, a second dissociation device 5, and a mass analyzer 6, which communicate in sequence. One or more ion optical devices 7 may be connected between the above components to focus, guide, or convey ions.

### <Ion Source>

The ion source 1 performs the ionization step S1 of ionizing a sample to obtain sample ions.

Examples of the ion source 1 include an ion source selected from the group consisting of: an electrospray ionization (ESI) source; an atmospheric pressure photoionization (APPI) source; an atmospheric pressure chemical ionization (APCI) source; a matrix-assisted laser desorption ionization (MALDI) source; a laser desorption ionization (LDI) source; an atmospheric pressure ionization (API) source; a desorption/ionization on silicon (DIOS) source; an electron impact ionization (EI) source; a chemical ionization (CI) source; a field ionization (FI) source; a field desorption (FD) source; an inductively coupled plasma (ICP) source; a fast atom bombardment (FAB) ion source; a liquid secondary ion mass spectrometry (LSIMS) ion source; an desorption electrospray ionization (DESI) source; a nickel-63 radioactive ion source; an atmospheric pressure matrix-assisted laser desorption ionization ion source; a thermal spray ion source; an atmospheric sampling glow discharge ionization (ASGDI) source; a glow discharge (GD) ion source; an impactor ion source; a direct analysis in real time (DART) ion source; a laser spray ionization (LSI) source; a sonic spray ionization (SSI) source; a matrix-assisted inlet ion source (MAII); a solvent assisted inlet ionization (SAII) source; Penning ionization source; laser ablation electrospray ionization (LAESI) source; and a He plasma (HePI) ion source. The ion source 1 is preferably an electrospray ionization source, a nanoelectrospray ionization source, a desorption electrospray ionization source, an atmospheric pressure chemical ionization source, an atmospheric pressure photoionization source, or a matrix-assisted laser desorption ionization source. In the present embodiment, the ion source 1 is preferably an electrospray ionization source.

### <Ion Mobility Spectrometer>

The ion mobility spectrometer 2 performs the ion mobility-based separation step S2 of separating the sample ions based on the difference in ion mobility and separating the target lipid ions from the sample ions.

Examples of the ion mobility spectrometer 2 include an ion mobility analysis device selected from the group consisting of: a drift tube ion mobility spectrometry (DTIMS) device; a differential mobility analysis (DMA) device, a field asymmetric-waveform ion-mobility spectrometry (FAIMS) device, a travelling wave ion mobility spectrometry (TW-IMS) device, a differential mobility spectrometry (DMS) device, a transversal modulation ion mobility spectrometry device, a trapped ion mobility spectrometry (TIMS) device, and a U-shaped ion mobility analyzer (UMA).

In the present embodiment, the ion mobility spectrometer 2 is preferably a U-shaped ion mobility analyzer. The ion mobility spectrometer 2 is more preferably a U-shaped ion mobility analyzer operating in a filtering mode. The device structure of the U-shaped ion mobility analyzer and the introduction of the filtering mode (or “filter mode”) suitable for the present embodiment can refer to Chinese patent CN113495112A.

The filtering mode is a mode in which non-target ions are filtered out and target ions are retained. The target ions are allowed to move along a specified path and pass through the filter. In other words, the filtering mode does not change an ion flow pattern of the target ions. As long as the input is a continuous ion flow with the target ions, the output will also be a continuous ion flow with the target ions. Not locally enriching or storing ions can avoid the loss of low-abundance ions caused by space charge effects, which is very suitable for lipidomics analysis research.

Ion mobility spectrometer 2 can provide data of a second dimension for tandem mass spectrometry analysis. Isomers can be distinguished based on differences in ion mobility. In some embodiments, the ion mobility spectrometry can also be used to identify the position of the carbon-carbon double bond or the differences between cis-trans isomerism and sn-isomerism.

### <First Dissociation Device>

The first dissociation device 3 performs the first dissociation step S3 of setting the dissociation energy of the first dissociation device 3 which is adjusted to break the first chemical bond with lower bond energy (such as the polar head group) in the target lipid ions and to keep the main chain of the target lipid ion complete.

Examples of the first dissociation device 3 include one or more dissociation devices selected from the group consisting of: a collision-induced dissociation (CID) device; a surface-induced dissociation (SID) device; an electron transfer dissociation (ETD) device; an electron capture dissociation (ECD) device; an electron collision-or-impact dissociation device; a photo ionization dissociation (PID) device; a laser-induced dissociation device; an infrared radiation-induced dissociation device; an ultraviolet radiation-induced dissociation device; a nozzle-separator interface dissociation device; an in-source dissociation device; an in-source collision-induced dissociation device; a heat-or-temperature source dissociation device; an electric field-induced dissociation device; a magnetic field-induced dissociation device; an ion-ion reaction dissociation device; an ion-molecule reaction dissociation device; an ion-atom reaction dissociation device; an ion-metastable ion reaction dissociation device; an ion-metastable molecule reaction dissociation device; and an electron ionization dissociation (EID) device.

In the present embodiment, the first dissociation device 3 is an in-source collision-induced dissociation device in which voltage is applied to a vacuum interface, such as an orifice, and the device is simpler. The voltage applied to the terminal electrodes is 10 eV to 70 eV. Within this voltage range, the polar head groups of a phospholipid or a sphingolipid can be efficiently removed.

### <Mass Filter>

The mass filter 4 performs the mass-based selection step S4 of selecting the target lipid ions, whose first chemical bond is broken, based on a mass number to obtain fragment ions.

Examples of the mass filter 4 include one or more mass filters selected from the group consisting of: a quadrupole mass filter; a 2D or linear quadrupole ion trap; a Paul or 3D quadrupole ion trap; a Penning ion trap; an ion trap; a magnetic sector mass filter; a time-of-flight mass filter; and a Wien filter.

### <Second Dissociation Device>

The second dissociation device 5 performs the second dissociation step S5 of further dissociating the fragment ions to break a second chemical bond of the fragment ions which has higher bond energy to obtain diagnostic ions.

Examples of the second dissociation device 5 include one or more dissociation devices selected from the group consisting of: a collision-induced dissociation (CID) device; a surface-induced dissociation (SID) device; an electron transfer dissociation (ETD) device; an electron capture dissociation (ECD) device; an electron collision-or-impact dissociation device; a photo ionization dissociation (PID) device; a laser-induced dissociation device; an infrared radiation-induced dissociation device; an ultraviolet radiation-induced dissociation device; a nozzle-separator interface dissociation device; an in-source dissociation device; an in-source collision-induced dissociation device; a heat-or-temperature source dissociation device; an electric field-induced dissociation device; a magnetic field-induced dissociation device; an ion-ion reaction dissociation device; an ion-molecule reaction dissociation device; an ion-atom reaction dissociation device; an ion-metastable ion reaction dissociation device; an ion-metastable molecule reaction dissociation device; and an electron ionization dissociation (EID) device.

Preferably, the second dissociation device 5 is a collision-induced dissociation device, and the dissociation energy is 30 eV to 70 eV. With this dissociation energy, a glycerol backbone and an aziridine ring can be selectively broken to reduce the generation of impurity ions, increase the peak intensity of diagnostic ions, and make the spectrum simpler and easier to read.

### <Mass Analyzer>

The mass analyzer 6 performs the mass analysis step S6 of performing mass analysis on the diagnostic ions.

Examples of the mass analyzer 6 include an mass analyzer selected from the group consisting of: a quadrupole mass analyzer; a 2D or linear quadrupole mass analyzer; a Paul or 3D quadrupole mass analyzer; a Penning trap mass analyzer; an ion trap mass analyzer; a magnetic sector mass analyzer; an ion cyclotron resonance (ICR) mass analyzer; a Fourier transform ion cyclotron resonance (FTICR) mass analyzer; an electrostatic mass analyzer, which is arranged to generate an electrostatic field with a quadrupole logarithmic potential distribution; a Fourier transform electrostatic mass analyzer; a Fourier transform mass analyzer; a time-of-flight mass analyzer; an orthogonal acceleration time-of-flight mass analyzer; and a linear acceleration time-of-flight mass analyzer. The mass analyzer 6 is preferably a high-resolution mass analyzer such as a time-of-flight mass analyzer.

The components of the ion mobility-tandem mass spectrometer according to the present embodiment are described above, but are not limited thereto. In other embodiments of the present disclosure, a separation device may also be provided at a preceding stage of the ion source 1, and the separation device may be one or more of liquid chromatography, gas chromatography, supercritical chromatography, capillary electrophoresis device, and paper chromatography.

### <Derivatization Reaction>

The method for determining a chemical structure of a lipid provided by the present embodiment further includes, before the ionization step S1, a derivatization reaction step of labeling the carbon-carbon double bond using a derivatization reaction. The derivatization reaction step may be implemented offline, that is, the step is implemented in a laboratory by an experimenter, or may be implemented online, that is, a sample and a reaction reagent are automatically introduced into a reactor to complete the reaction. The implementation manner of the derivatization reaction step is not limited in the present embodiment.

The derivatization reaction may be any derivatization reaction that can convert a carbon-carbon double bond into an easily dissociable group, such as an aziridination reaction, an epoxidation reaction, and a singlet oxygen-ene reaction. More specifically, the derivatization reaction may be, for example, Paternò-Büchi reaction, Diels-Alder reaction, aza-Prilezhaev reaction, and a singlet oxygen-ene reaction. In the present embodiment, there is no limitation on the reaction type used.

In the present embodiment, the aza-Prilezhaev reaction is used as the derivatization reaction. The reaction mechanism of the aza-Prilezhaev reaction is as follows.

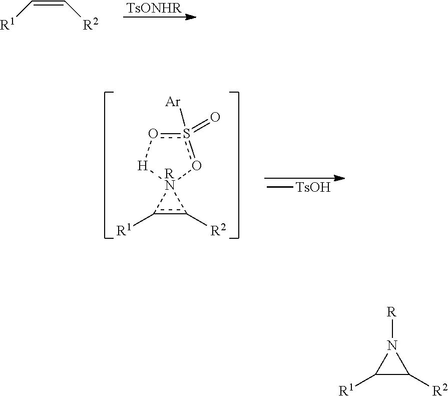

The derivatization reagent is a mass marker dissolved in an acidic solvent. Specifically, the mass marker is N-Boc-O-tosylhydroxylamine (CAS: 105838-14-0), the acidic reagent is hexafluoroisopropanol, and the lipid and the mass marker react under a heating condition of 20° C. to 100° C. for 10 minutes or longer to aziridine the carbon-carbon double bond.

The dissociation energy for breaking the glycerol backbone or the aziridine ring is higher than the dissociation energy for breaking the polar head group, but is still lower than the dissociation energy for breaking other chemical bonds, such as a carbon-carbon bond of a fatty acyl chain. Therefore, no matter in the first dissociation step S3 or the second dissociation step S5, the first chemical bond (such as the polar head group) and the second chemical bond (such as the glycerol backbone and the aziridine ring) which are mainly expected to be broken can be broken without a need to apply excessive dissociation energy to the compound obtained after the derivatization reaction, so that the problem that the spectrum is too complex due to the fact that excessive dissociation energy needs to be applied is avoided, and the signal intensity of the diagnostic ions can be further improved.

In addition, the carbon-carbon double bond is derivatized into a more rigid structure such as an aziridine ring or an epoxy ring, so that the structural differences between different molecules can be further amplified, and the resolution capability of the ion mobility spectrum is improved.

### <Ion Reaction Process>

The method for determining a chemical structure of a lipid according to the present embodiment is described below by taking the PC (18:1/16:0), which is used as target lipid ions, as an example.

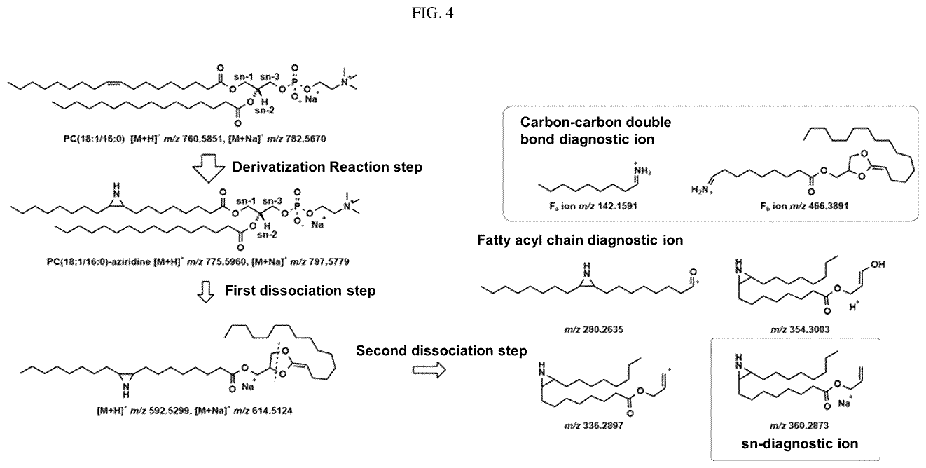

FIG. 4 shows a specific reaction process of PC (18:1/16:0) in the present embodiment. Referring to FIG. 4, an original sample is first pretreated, i.e., derivatized, to convert a carbon-carbon double bond of an unsaturated lipid in the original sample into an aziridine ring.

In the ionization step S1, the molecules of each component can be converted into sample ions with positive charge, and then mass spectrometry analysis is performed in a positive ion mode. PC (18:1/16:0) contained in the sample was ionized after aziridine formation to obtain target lipid ions. The positive ions obtained by hydrogenation are target lipid ions with a mass number of 775.6, and the positive ions obtained by the addition of sodium are the target lipid ions with a mass number of 797.6.

Next, in the ion mobility-based separation step S2, target lipid ions can be separated from the sample ions within a specific time period of an analysis cycle and conveyed to the subsequent stage by using an ion mobility spectrometer. Alternatively, the ion mobility spectrometer may also be configured in a filtering mode, that is, the target lipid ions in the sample ions are continuously screened out and conveyed to the subsequent stage.

Then, in the first dissociation step S3, the target lipid ions are dissociated to remove the polar head group of PC (18:1/16:0), that is, a phosphorylcholine group. After the phosphorylcholine group is removed, a fragment ion having a 1,3-dioxolane structure as shown in FIG. 4 can be obtained.

In the second dissociation step S4, the fragment ion having the 1,3-dioxolane structure may be broken at the position of 1,3-dioxolane structure, and the aziridine ring obtained by the derivatization reaction may also be broken, thereby forming a plurality of diagnostic ions. A part of the diagnostic ions may be used to identify the position of the carbon-carbon double bond in the fatty acyl chain, that is, the C═C position diagnostic ions marked in FIG. 4. A part of the diagnostic ions may be used to identify the sn-position of the carbon-carbon double bond, that is, the sn-position diagnostic ions marked in FIG. 4. A part of the diagnostic ions may be used to determine the information of the fatty acyl chain where the carbon-carbon double bond is located, that is, the fat chain diagnostic ions marked in FIG. 4.

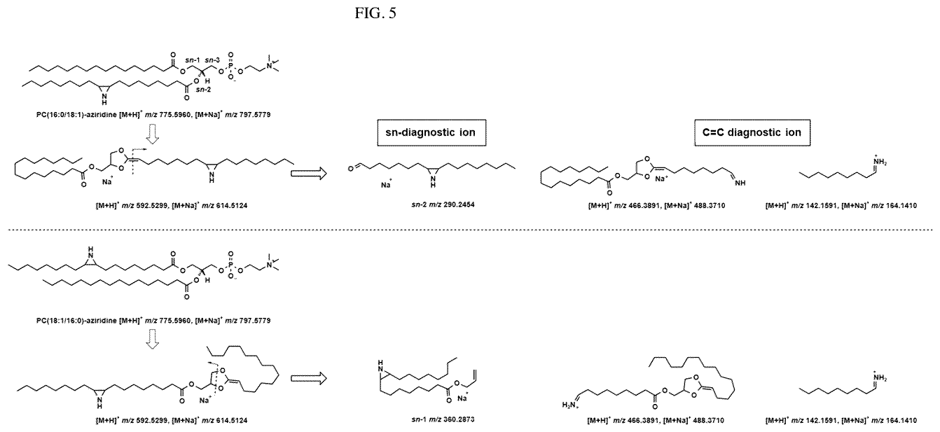

FIG. 5 is a schematic diagram illustrating why a sn-position of a carbon-carbon double bond can be identified based on diagnostic ions with a mass number of m/z=290 or 360 [M+Na⁺].

Referring to FIG. 5, it can be seen that when the carbon-carbon double bond is located at different sn-positions, the fragment ions and diagnostic ions produced by dissociation will be different. PC (16:0/18:1) with a carbon-carbon double bond at the sn-2 position can be identified based on a diagnostic ion with a mass number of 290 [M+Na⁺]. PC (18:1/16:0) with a carbon-carbon double bond at the sn-1 position can be identified based on a diagnostic ion with a mass number of 360 [M+Na⁺].

Based on the above reaction process, it can be seen that the method for determining a chemical structure of a lipid provided in the present embodiment can simultaneously determine a position of a carbon-carbon double bond, a fatty acyl chain composition, and a sn-position in a fatty acyl chain of phospholipids or sphingolipids through a single injection, and has excellent analysis efficiency.

Some main steps of the method for determining a chemical structure of a lipid have been described above, but the present disclosure is not limited thereto. In other embodiments of the present disclosure, the method may further include other steps.

For example, before performing the first dissociation step S3 and the second dissociation step S5, a first pre-scan step may be performed to perform mass analysis on the sample ions not subjected to the first dissociation step S3 and the second dissociation step S5. The first pre-scan step may be used to find target lipid ions, that is, target lipid ions with a mass number of 775 [M+H⁺] or 797 [M+Na⁺] in the present embodiment, and then further determine isomers of the target lipid ions.

For example, after performing the first dissociation step S3 and before performing the second dissociation step S5, a second pre-scan step may be performed to perform mass analysis on the sample ions subjected to only one dissociation. The second pre-scan step can be used to determine the appropriate dissociation energy so that the target lipid ions can be dissociated in a manner that only the polar head group is broken as much as possible, to reduce or avoid the generation of redundant fragment ions. Thus, signal intensity is increased, and the spectrum is easier to read.

### Experimental Results

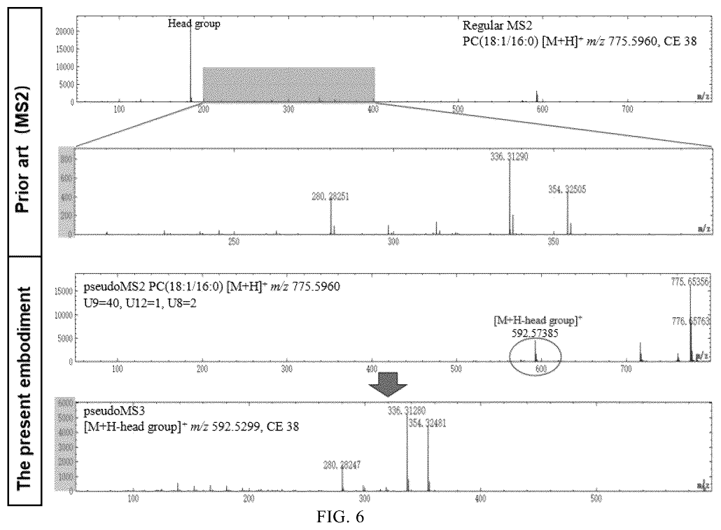

FIG. 6 is a comparison chart of measurement results obtained by the common tandem mass spectrometry MS2 method in the prior art and measurement results obtained by the method provided in the present embodiment.

In the two mass spectra corresponding to the prior art of FIG. 6, the upper figure is an MS2 spectrum, and the lower figure is a spectrum obtained by enlarging a range framed in the upper figure. In the two mass spectra corresponding to the measurement results of the present embodiment of FIG. 6, the upper figure is an MS1 spectrum (labeled pseudoMS2 means that the IMS and the first dissociation device 3 are MS1, the same below), and the lower figure is an MS2 spectrum obtained by further dissociating the ions with a mass number of 592.

Referring to FIG. 6, it can be seen that the peak intensity of diagnostic ions in the MS2 spectrum of the common tandem mass spectrum is about within a range of 700 to 800 due to the interference of the polar head group, while the peak intensity of the diagnostic ions obtained by the IMS-CID-MS/MS method provided by the present embodiment can reach a range of 5000 to 6000, which greatly improves the signal intensity (about 7 times to 8 times) and improves the analysis sensitivity.

The above are only preferred embodiments of the present disclosure and are not intended to limit the present disclosure, and any modifications, equivalent substitutions, and improvements made within the spirit and principle of the present disclosure shall fall within the scope of the present disclosure.

1. A method for determining a chemical structure of a lipid, the method comprising:
an ionization step of ionizing a sample to obtain sample ions;
an ion mobility-based separation step of separating target lipid ions from the sample ions based on ion mobility;
a first dissociation step of dissociating the target lipid ions with dissociation energy adjusted to break a first chemical bond of the target lipid ions;
a mass-based selection step of selecting the target lipid ions, whose first chemical bond is broken, based on a mass number to obtain fragment ions;
a second dissociation step of dissociating the fragment ions to at least break a second chemical bond of the fragment ions which has bond energy higher than the first chemical bond, to obtain diagnostic ions; and
a mass analysis step of performing a mass analysis on the diagnostic ions.

2. The method for determining a chemical structure of a lipid according to claim 1, wherein the lipid is an unsaturated lipid having a carbon-carbon double bond in a fatty acyl chain, and the method is used for identifying a position of the carbon-carbon double bond in the fatty acyl chain and a sn-position of the fatty acyl chain.

3. The method for determining a chemical structure of a lipid according to claim 2, further comprising, before the ionization step:
a derivatization reaction step of labeling the carbon-carbon double bond using a derivatization reaction.

4. The method for determining a chemical structure of a lipid according to claim 3, wherein the lipid is a phospholipid or a sphingolipid, the first chemical bond is a polar head group of the phospholipid or a polar head group of the sphingolipid, and the second chemical bond is a chemical bond generated from derivatization of the carbon-carbon double bond.

5. The method for determining a chemical structure of a lipid according to claim 3, wherein the derivatization reaction is an aziridination reaction, an epoxidation reaction, a Paternò-Büchi reaction, a singlet oxygen-ene reaction, or a Diels-Alder reaction.

6. The method for determining a chemical structure of a lipid according to claim 1, wherein the lipid is a fatty acid, a glycerolipid, a glycerophospholipid, a sphingolipid, a sterol lipid, a prenol lipid, a saccharolipid, or a polyketide.

7. The method for determining a chemical structure of a lipid according to claim 1, further comprising:
a first pre-scan step of performing a mass analysis on the sample ions that are not subjected to the first dissociation step and the second dissociation step.

8. The method for determining a chemical structure of a lipid according to claim 1, further comprising:
a second pre-scan step of performing a mass analysis on the sample ions that are subjected to only one dissociation.

9. An ion mobility-tandem mass spectrometer comprising:
an ion source configured to ionize a sample to obtain sample ions;
an ion mobility spectrometer configured to separate target lipid ions from the sample ions;
a first dissociation device configured to dissociate the target lipid ions, in which dissociation energy is adjusted to break a first chemical bond of the target lipid ions;
a mass filter configured to select the target lipid ions, whose first chemical bond is broken, to obtain fragment ions;
a second dissociation device configured to dissociate the fragment ions to at least break a second chemical bond of the fragment ions which has bond energy higher than that of the first chemical bond to obtain diagnostic ions; and
a mass analyzer configured to perform a mass analysis on the diagnostic ions.

10. The ion mobility-tandem mass spectrometer according to claim 9, wherein the ion mobility spectrometer is a U-shaped ion mobility spectrometer.

11. The ion mobility-tandem mass spectrometer according to claim 10, wherein the U-shaped ion mobility spectrometer operates in a filter mode.

12. The ion mobility-tandem mass spectrometer according to claim 9, wherein the mass filter is a quadrupole or an ion trap.

13. The ion mobility-tandem mass spectrometer according to claim 9, wherein the mass analyzer is a time-of-flight mass analyzer, a Fourier transform mass spectrometer, a quadrupole mass analyzer, an ion trap mass analyzer, or a magnetic mass spectrometer.

14. The ion mobility-tandem mass spectrometer according to claim 9, wherein the ion source is an electrospray ionization source, a nanoelectrospray ionization source, a desorption electrospray ionization source, an atmospheric pressure chemical ionization source, an atmospheric pressure photoionization source, or a matrix-assisted laser desorption ionization source.

15. The ion mobility-tandem mass spectrometer according to claim 9, wherein the first dissociation device and/or the second dissociation device are/is one or more of a high-energy collision dissociation device, a collision-induced dissociation device, an oxygen-attachment dissociation device, a hydrogen-attachment dissociation device, an electron-capture dissociation device, a radical-directed dissociation device, an ultraviolet photodissociation device, and a charge-remote fragmentation device.

16. The ion mobility-tandem mass spectrometer according to claim 15, wherein the first dissociation device is a collision-induced dissociation device and has a terminal electrode voltage of 10 eV to 70 eV, and the second dissociation device is a collision-induced dissociation device and has dissociation energy of 30 eV to 70 eV.

17. The ion mobility-tandem mass spectrometer according to claim 9, further comprising: a separation device disposed at a preceding stage of the ion source, wherein the separation device is one or more of a liquid chromatograph, a gas chromatograph, a supercritical fluid chromatograph, a capillary electrophoresis device, and a paper chromatograph.
